(19) United States
(12) Patent Application Publication
(10) Pub. No.: US 2026/0115406 A1
(43) Pub. Date: Apr. 30, 2026

(54) ARRANGEMENT AND PROCESS FOR ARTIFICIAL VENTILATION OF A PATIENT WITH REDUCED RISK OF CONDENSATION

(71) Applicant: Drägerwerk AG & Co. KGaA, Lübeck (DE)

(72) Inventors: Ralf HEESCH, Lübeck (DE); Klaus RADOMSKI, Lübeck (DE)

(21) Appl. No.: 19/369,948

(22) Filed: Oct. 27, 2025

(30) Foreign Application Priority Data
Oct. 28, 2024 (DE) 10 2024 131 337.4

(51) Int. Cl. A61M 16/10; A61M 16/00; A61M 16/12; G16H 40/63

(52) U.S. Cl. CPC A61M 16/1075; A61M 16/024; A61M 16/104; A61M 16/12; G16H 40/63; A61M 2205/3368

(57) ABSTRACT

A ventilation system and a ventilation process artificially ventilate a patient. A control arrangement and a control process control a ventilation arrangement belonging to the ventilation system. An anesthetic dispenser unit (38) provides an anesthetic gas mixture (Ng) with a predetermined target concentration (con_{req}) of an anesthetic (Nm). A ventilator (12) conveys a breathable gas mixture with the anesthetic gas mixture (Ng) to a patient-side coupling unit. The target concentration (con_{req}) is specified so that it is at most equal to a calculated upper concentration threshold (conₘₐₓ). The higher the temperature in the anesthetic dispenser unit (38), in the ventilator (12) and/or in an environment, the greater is the upper concentration threshold (conₘₐₓ). The risk of anesthetic (Nm) condensing is reduced.

### CROSS REFERENCE TO RELATED APPLICATIONS

This application claims the benefit of priority under 35 U.S.C. § 119 of German Application 10 2024 131 337.4, filed Oct. 28, 2024, the entire contents of which are incorporated herein by reference.

### TECHNICAL FIELD

The invention relates to a ventilation system (also known as a respiration system or respiratory system) and a ventilation process for artificial ventilation of a patient. Furthermore, the invention relates to a control arrangement and a control process for controlling a ventilation arrangement, wherein the controlled ventilation arrangement is a component of such a ventilation system.

### BACKGROUND

Many ventilation arrangements known from the state of the art are configured to artificially ventilate a patient while the patient is anesthetized or at least sedated. During artificial ventilation, a patient-side coupling unit is arranged in and/or on the patient's body. A ventilator conveys a breathable gas mixture comprising oxygen and an anesthetic gas mixture to the patient-side coupling unit. The anesthetic gas mixture comprises at least one anesthetic. Often a desired (target) concentration or a desired volume flow or mass flow of the anesthetic in the anesthetic gas mixture are specified. The actual concentration or the actual volume flow or mass flow should come as close as possible to this specification, ideally corresponds to (coincides with) this specification.

### SUMMARY

It is an object of the invention to provide a ventilation system and a ventilation process which are intended to convey a breathable gas mixture with an anesthetic gas mixture to a patient-side coupling unit and to provide the anesthetic gas mixture with a desired concentration of anesthetic more reliably than known ventilation systems and ventilation processes. Furthermore, it is another object of the invention to provide a control arrangement and a control process for controlling a ventilation arrangement, wherein the control arrangement and the control process are configured to ensure that the anesthetic gas mixture is provided with a desired concentration of anesthetic more reliably than by known control arrangements and control processes.

The object is attained, and the problem is solved by a ventilation system with features as disclosed herein, by a control arrangement with features as disclosed herein, by a control process with features as disclosed herein and by a ventilation process with features as disclosed herein. Advantageous embodiments are given in the specification, drawings and claims. Advantageous embodiments of the ventilation system according to the invention are, where appropriate, also advantageous embodiments of the control arrangement according to the invention, the control process according to the invention and the ventilation process according to the invention and vice versa.

The ventilation system according to the invention comprises a ventilation arrangement. The ventilation arrangement is configured to artificially ventilate a patient. A patient is artificially ventilated by the ventilation process according to the invention. The ventilation process according to the invention is carried out using a ventilation arrangement according to the invention. A ventilation arrangement according to the invention is controlled by the control arrangement according to the invention and the control process according to the invention. The ventilation arrangement and the control arrangement are part of the ventilation system. The control arrangement can be a part of the ventilation arrangement and/or be spatially remote from the ventilation arrangement. When performing the ventilation process, the steps of the control process are carried out.

A patient-side coupling unit of the ventilation arrangement can be arranged in and/or on the body of a patient who is to be artificially ventilated. A breathing mask on a patient's face and a tube in the patient's body are two examples of a patient-side coupling unit. The control process and the ventilation process are carried out while the patient-side coupling unit is placed in and/or on the patient's body.

The ventilation arrangement further comprises an anesthetic dispenser unit, optionally several anesthetic dispenser units, and a ventilator with a ventilator housing. A receptacle is embedded in the ventilator housing, optionally several receptacles are embedded in the ventilator housing. The or each anesthetic dispenser unit is permanently or at least temporarily inserted into the or a respective receptacle in the ventilator housing. Preferably, the anesthetic dispenser unit can be inserted into the receptacle and removed again from this receptacle. Preferably, exactly one anesthetic dispenser unit is inserted or can be inserted into the receptacle or one receptacle at any time, except in a resting state. If the ventilation arrangement comprises several anesthetic dispenser units, each anesthetic dispenser unit is preferably inserted into a respective one receptacle. The control process and the ventilation process are performed while the anesthetic dispenser unit or an anesthetic dispenser unit is inserted into the or at least one receptacle.

The or each anesthetic dispenser unit is configured to generate (produce) and supply (provide) an anesthetic gas mixture. This anesthetic gas mixture comprises at least one anesthetic and, in one embodiment, additionally oxygen. The anesthetic dispenser unit is configured to generate the anesthetic gas mixture when it is inserted into the or a receptacle in the ventilator housing. Optionally, several anesthetic dispenser units are inserted simultaneously in respective receptacles, and each anesthetic dispenser unit generates an anesthetic gas mixture with an anesthetic, optionally two anesthetic gas mixtures with two different anesthetics and/or with two different anesthetic concentrations.

The ventilator is configured to generate a breathable gas mixture. The breathable gas mixture comprises oxygen and the anesthetic gas mixture generated and provided by the or at least one inserted and used anesthetic dispenser unit. To generate the breathable gas mixture, the ventilator uses the anesthetic gas mixture and preferably at least one gas from a supply unit, for example from a supply port or a supply container. The ventilator is configured to convey the breathable gas mixture to the patient-side coupling unit. Preferably, the ventilator performs a sequence of ventilation strokes and conveys in each ventilation stroke a respective quantity of the breathable gas mixture to the patient-side coupling unit. The patient can inhale the breathable gas mixture that is conveyed to the patient-side coupling unit, or it is conveyed into the patient's body.

A signal-processing control unit belongs to the ventilation system according to the invention, optionally to the ventilation arrangement according to the invention, and to the control arrangement according to the invention. The control unit is configured to calculate a target concentration of the anesthetic in the anesthetic gas mixture. The control process according to the invention and the ventilation process according to the invention are carried out using such a control unit. The calculated target (desired) concentration specifies the concentration at which the anesthetic or an anesthetic should occur in the anesthetic gas mixture generated by the anesthetic dispenser unit. The target concentration therefore specifies a specification for the anesthetic dispenser unit. If the anesthetic gas mixture contains at least two anesthetics, the control unit preferably calculates a respective target concentration for each anesthetic. These target concentrations can differ from one another.

The control unit is configured to control the or each inserted anesthetic dispenser unit. The objective of this control is to ensure that the anesthetic dispenser unit provides the anesthetic gas mixture as follows: The or each anesthetic is present in the anesthetic gas mixture with the calculated respective target concentration. This objective can usually only be achieved approximately.

The ventilation system and the control arrangement further comprise at least one temperature sensor from a temperature sensor group. The control process and the ventilation process are performed using at least one such temperature sensor. The or every comprised temperature sensor is selected from the group consisting of the following three temperature sensors:
an ambient temperature sensor,
a temperature sensor on the anesthetic dispenser side and
a temperature sensor on the ventilator side.

Because the at least one temperature sensor is selected from the group consisting of three different temperature sensors and at least one is actually used, with regard to the temperature sensors 2\hat{ }3−1=7 different configurations of a ventilation system according to the invention and a control arrangement according to the invention are possible. It is possible that the ventilation arrangement or the control arrangement comprises a further sensor that does not belong to the temperature sensor group, for example a pressure sensor or a volume flow sensor or a temperature sensor that measures the temperature of the breathable gas mixture.

The ambient temperature sensor is configured to measure the temperature in an environment of the ventilation arrangement and thus of the ventilation system at least once, preferably several times, so that a time course of the ambient temperature is measured. The ambient temperature sensor or at least the measuring position of the ambient temperature sensor can be spatially separate from the ventilation arrangement.

It is also possible that the ambient temperature sensor is located in the ventilator. In one embodiment, the ambient temperature sensor in the ventilator measures a difference between the temperature in the ventilator and the temperature in an environment of the ventilator.

In another embodiment, the ambient temperature sensor measures a temperature in the ventilator. A deviation between the temperature in the ventilator and the ambient temperature is predetermined. This deviation can depend on the temperature in the ventilator and/or on the previous period of use of the ventilator and was preferably determined empirically in advance. It is often justified to assume that this deviation does not depend on the ambient temperature. To derive the ambient temperature, the ambient temperature sensor combines the measured temperature in the ventilator with the specified deviation. Optionally, a functional relationship is determined in advance that describes the deviation as a function of the measured temperature in the ventilator and/or the period of use of the ventilator. This functional relationship is then applied to the measured temperature in the ventilator.

The anesthetic dispenser temperature sensor, that is the temperature sensor on the anesthetic dispenser side, is configured to measure at least once a temperature at an anesthetic dispenser measuring position, preferably several times. The anesthetic dispenser measuring position is located in or on the anesthetic dispenser unit. Preferably, the anesthetic dispenser measuring position is in thermal contact with a surface of the anesthetic dispenser unit. When the anesthetic dispenser unit is inserted in a receptacle, this surface faces the housing of the ventilator. Preferably, the anesthetic dispenser temperature sensor is a component of the anesthetic dispenser unit, but it can also be arranged inside the ventilator. It is possible that the ventilation system comprises several anesthetic dispenser units and that each of these units comprises a temperature sensor on the anesthetic dispenser side or provides at least one anesthetic dispenser measuring position.

The ventilator temperature sensor is configured to measure a temperature at a ventilator measuring position at least once, preferably several times. The ventilator measuring position is located in or on the ventilator, preferably in the vicinity of the or a receptacle in the housing. Preferably, the ventilator measuring position is in thermal contact with a surface of the receptacle in the housing of the ventilator. When the anesthetic dispenser unit is inserted, this surface faces the anesthetic dispenser unit. Preferably, the ventilator temperature sensor is a component of the ventilator. It is possible that several receptacles, each for one anesthetic dispenser unit, are embedded in the housing and that the ventilation system and the control arrangement each comprise a ventilator temperature sensor for each receptacle or each provide at least one ventilator measuring position.

Note: The phrase is used that a sensor is configured to measure a physical quantity, for example a temperature at a measuring position. This phrase means that the sensor is configured to measure the physical quantity directly or another quantity that correlates with the quantity to be measured and is therefore an indicator of the physical quantity to be measured. The measurement provides at least one value of the physical quantity sought.

The term “measurable temperature” is used below. This refers to a temperature at one of the three measurement positions just mentioned. The control unit is configured to calculate the target concentration as a function of at least one measurable temperature. Because the ventilation system and the control arrangement comprise at least one temperature sensor of the temperature sensor group, at least one measurable temperature is actually measured. A measurable temperature is either measured by an actual temperature sensor of the temperature sensor group, or a standard value (default value) for a measurable temperature is used. Or at least one measurable temperature is not taken into account to calculate the target concentration.

A functional relationship that can be evaluated by a computer is stored on a data memory. The data memory belongs to the ventilation system, optionally to the ventilation arrangement, and to the control arrangement. The functional relationship can also be part of a program that the control unit is configured to execute. The control process and the ventilation process are carried out using such a functional relationship.

The functional relationship specifies an upper concentration threshold for the concentration of the anesthetic in the anesthetic gas mixture as a function of at least one measurable temperature, optionally of several measurable temperatures. With exactly one measurable temperature in the functional relationship, each value of the occurring measurable temperature thus leads to one value for the upper concentration threshold. With at least two measurable temperatures in the functional relationship, each combination of values of the occurring measurable temperatures leads to one value for the upper concentration threshold. The functional relationship is configured as follows: If the or one measurable temperature occurring in the functional relationship increases and, in the case of several measurable temperatures, the or each other measurable temperature remains the same (constant), the upper concentration threshold becomes greater or remains at least the same. In other words: The functional relationship is monotonically increasing in the or each argument (one measurable temperature).

As already explained, the control unit is configured to calculate a target concentration for the anesthetic or an anesthetic in the anesthetic gas mixture. The control unit is configured to perform the following steps when calculating the target concentration:
The control unit applies the functional relationship to at least one measured value of each measurable temperature, preferably to the most recently measured value, optionally to several values. If several measurable temperatures are actually measured, the control unit applies the functional relationship on at least one respective measured value of every measurable temperature.
The control unit derives a value for the upper concentration threshold through the application.
The control unit calculates the target concentration in such a way that the target concentration is at most equal to the derived value for the upper concentration threshold. This means that the target concentration depends on the measured temperature.

It is possible that the functional relationship refers to a specific measurable temperature, but the ventilation arrangement for this temperature does not actually include a temperature sensor or this temperature sensor is defective or switched off. In this case, the control unit preferably uses a standard value, particularly preferably a predefined lower threshold or a lowest possible value for this measurable temperature. If the lower threshold is used, “you are on the safe side”, which is described below.

Several advantages of the invention are described below.

It is desirable that only gaseous anesthetic flows as part of the anesthetic gas mixture and as part of the breathable gas mixture from the or at least one inserted anesthetic dispenser unit through a fluid guide unit to the patient-side coupling unit. In many cases, this desired situation is ensured by a sufficiently high ambient temperature and optionally by heating this fluid guide unit.

Every anesthetic has a saturation concentration (saturated vapor concentration). If the concentration of anesthetic in a gas mixture is above this saturation concentration, some of the anesthetic will usually condense. As a rule, the saturation concentration varies from anesthetic to anesthetic and also depends on the temperature of the gas mixture. The higher the temperature of the gas mixture is, the higher is the saturation concentration of a particular anesthetic. Typically, the saturation concentration of an anesthetic at a particular temperature is a known property of that anesthetic.

The undesirable situation can occur that gaseous anesthetic condenses on its way from the anesthetic dispenser unit to the patient-side coupling unit, for example on an inner wall of a used fluid guide unit. In particular, this undesirable situation can occur at the start of artificial ventilation. One cause is as follows: The anesthetic dispenser unit is kept in stock in a relatively cool storage room and inserted into the or a receptacle before the start of artificial ventilation. After the anesthetic dispenser unit has been inserted into the or a receptacle, a certain warm-up phase inevitably elapses due to its thermal mass until the anesthetic dispenser unit has reached at least the ambient temperature. In addition, the ventilator can also be relatively cool at the start of artificial ventilation. One consequence of this is that the concentration of anesthetic is above the saturation concentration at at least one point in the ventilation arrangement.

One reason why condensation of anesthetic is undesirable is described in the following. Condensation often leads to the following undesirable event: A ventilation arrangement often includes an anesthetic sensor. The anesthetic sensor is configured to measure the concentration of an anesthetic in a gas mixture. The gas mixture is in particular the anesthetic gas mixture or the breathable gas mixture. The measured actual anesthetic concentration is used, for example, for closed-loop control. In particular in this embodiment, the process of gaseous anesthetic condensing, for example on a wall of a fluid guide unit or on another surface of the ventilation arrangement, can have the following undesirable consequences: The anesthetic sensor does not correctly measure the concentration of the anesthetic in the anesthetic gas mixture and/or in the breathable gas mixture, but instead measures an incorrect anesthetic concentration, in particular one that is too low. This in turn can lead, particularly in a closed-loop control, to the respirable gas mixture having an incorrect, in particular an undesirably high, anesthetic concentration.

The invention reduces the risk of this undesirable situation occurring. The invention achieves this effect in particular by the control unit calculating a value for the upper concentration threshold and ensuring that the target concentration and preferably also the actual concentration of the anesthetic is not above this calculated value. This calculated value depends on at least one measurable temperature. The higher the measurable temperature is, the higher is the calculated value for the upper concentration threshold. At a higher measurable temperature, the risk of gaseous anesthetic condensing is lower than at a lower measurable temperature. Each measurable temperature within the meaning of the invention influences the temperature of the anesthetic gas mixture or depends on the temperature of the anesthetic gas mixture.

The invention can be used in combination with at least one heater, wherein the heater heats the anesthetic dispenser unit or an anesthetic dispenser unit and/or the ventilator. However, the invention avoids the need to heat a segment or even the entire fluid guide unit from the anesthetic dispenser unit to the patient-side coupling unit in order to prevent unwanted condensation. Even in combination with a heater, the invention takes into account the fact that both an anesthetic dispenser unit and a ventilator each have a thermal mass and therefore a certain amount of time elapses before they are heated.

The invention can be used in conjunction with a temperature sensor that measures the temperature of the anesthetic gas mixture or the breathable gas mixture generated. However, the invention does not require that such a temperature sensor is used. In some cases, it is more difficult to measure the temperature of a gas than the temperature at the measuring positions of the temperature sensors according to the invention. Furthermore, a temperature sensor alone does not prevent unwanted condensation.

The following configuration would also be conceivable in order to prevent undesired condensation: It is ensured that the target concentration is below the saturation concentration of the lowest possible temperature, wherein the actual temperature of the anesthetic gas mixture or the breathable gas mixture is always at least equal to this lowest possible temperature during use. However, this configuration restricts the possible uses of the ventilation system and the ventilation arrangement. This is because a higher target concentration can often not be achieved even at a higher temperature of the anesthetic gas mixture or the respirable gas mixture, although at this temperature the higher target concentration is still below the saturation concentration and therefore condensation is not to be feared. Rather, according to the invention, the achievable target concentration depends on at least one measurable and actually measured temperature, in such a way that the greater the measurable temperature, the greater the target concentration.

In many cases, the invention can be implemented on an existing ventilation arrangement by adapting software on a control unit of the ventilation arrangement wherein the control unit is usually also already present. The physical components required to implement the invention are often already present. In particular, at least one temperature sensor of the temperature sensor group is often already present. In particular, the invention does not require the addition of a heater or a temperature sensor for a gas mixture.

In one embodiment, the ventilation system and the control arrangement comprise two temperature sensors from the temperature sensor group, namely the anesthetic dispenser temperature sensor, on the anesthetic dispenser side, and the ventilator temperature sensor, on the ventilator side, but not necessarily an ambient temperature sensor. In the control process and the ventilation process, according to this embodiment, the temperature at the anesthetic dispenser measurement position and the temperature at the ventilator measurement position are measured, but not necessarily the ambient temperature. In this embodiment, the ventilation system and the control arrangement therefore do not necessarily comprise the ambient temperature sensor. However, an ambient temperature sensor can be used additionally, in particular if there is a possibility that the ambient temperature at the start of an operation is lower than the temperature of the ventilator and the temperature of the or each inserted anesthetic dispenser unit. In this situation, the temperature of the ventilator and that of the anesthetic dispenser unit often do not increase due to a higher ambient temperature, but remain the same or even decrease.

According to this embodiment, the functional relationship preferably specifies the upper concentration threshold as a function of the temperature at the anesthetic dispenser measuring position and the temperature at the ventilator measuring position, i.e. as a function of at least two measurable temperatures.

According to this embodiment, the control unit is configured to perform the following steps when deriving the value for the upper concentration threshold: The control unit applies the functional relationship to the measured value of the temperature at the anesthetic dispenser measuring position and to the measured value of the temperature at the ventilator measuring position.

In many cases, this configuration makes it possible to derive a larger value for the upper concentration threshold than if only or instead the measured ambient temperature had been used. At the same time, the risk of anesthetic condensation is generally not significantly increased.

In one implementation of this embodiment, an aggregation rule is specified. The aggregation rule specifies an aggregated temperature as a function of the temperature at the anesthetic dispenser measuring position and the temperature at the ventilator measuring position, optionally also as a function of the ambient temperature. The functional relationship describes the upper concentration threshold as a function of the aggregated temperature. The aggregation rule is configured as follows: The smaller a measurable temperature occurring in the aggregation rule is, the smaller is the aggregated temperature. The aggregation rule is, for example, the minimum (the smaller of the two temperatures) or a weighted average. The functional relationship then specifies the upper concentration threshold as a function of the aggregated temperature. The control unit applies the aggregation rule to the measured temperature values and then applies the functional relationship to the resulting value of the aggregated temperature.

According to the invention, the ventilation system and the control arrangement comprise at least one temperature sensor from the temperature sensor group. An embodiment was described above in which a temperature sensor on the anesthetic dispenser side and a temperature sensor on the ventilator side are used. The embodiment described below can be used in combination with these two temperature sensors, but eliminates the need to use such a temperature sensor. Rather, the embodiment described below only requires the ambient temperature sensor as a temperature sensor. According to this embodiment, the ambient temperature is measured before or during the control process and the ventilation process.

The background to this is as follows: After a warm-up phase or even a cool-down phase, the temperature of the ventilator and the temperature of the or each inserted anesthetic dispenser unit deviate only slightly from the ambient temperature. If the target concentration after the warm-up phase is lower than the saturation concentration at the ambient temperature, the risk of anesthetic condensation is relatively low. The duration of the warm-up phase can be specified relatively reliably in many cases, optionally as a function of the ambient temperature.

The embodiment described below applies to the situation in which the or each anesthetic dispenser unit can be inserted into the or a receptacle and removed again from the receptacle. When the anesthetic dispenser unit is inserted into the receptacle and the ventilation arrangement is used, both the temperature of the inserted anesthetic dispenser unit and the temperature of the ventilator automatically adjust to the ambient temperature. After a warm-up or cool-down phase, these three temperatures are therefore approximately the same. This applies provided that the anesthetic gas mixture is not heated. If the anesthetic gas mixture is heated, its saturation concentration is even higher.

In one embodiment, a relatively low standard value is specified for the upper concentration threshold. This standard value is used in the warm-up phase. At the end of the warm-up phase, a value is used for the upper concentration threshold which value depends on the measured ambient temperature and which the control unit has calculated according to the invention. As a rule, the calculated value is greater than the specified standard value. The following describes an implementation of how this warm-up phase is detected.

According to this embodiment, a system clock and an insertion sensor are used for each receptacle. The system clock can be a component of the control unit. The insertion sensor for a receptacle is configured to detect whether an anesthetic dispenser unit is inserted in this receptacle or not. The system clock is configured to measure the time. The control unit is configured to measure an insertion time span (anesthetic dispenser unit duration of deployment). This insertion time span has elapsed since the anesthetic dispenser unit was inserted without the anesthetic dispenser unit having been removed from the receptacle. To measure the insertion time span, the control unit uses a signal from the insertion sensor and a signal from the system clock.

In a possible further development of this embodiment, the standard value for the upper concentration threshold described above is used in the warm-up phase. In many cases, an alternative further development described below leads to a higher value for the upper concentration threshold without a significantly greater risk of anesthetic condensation.

According to this alternative embodiment, a functional relationship is specified which specifies the upper concentration threshold as a function of the ambient temperature and also as a function of the insertion time span. The functional relationship is specified as follows: If the insertion time span remains constant, the higher the ambient temperature, the higher the upper concentration threshold. Conversely, if the ambient temperature remains constant, the longer the insertion time span, the greater the upper concentration threshold. This alternative embodiment takes advantage of the following fact: As a rule, at the beginning of a use, the ambient temperature is greater than the temperature of the ventilator and the temperature of the or each anesthetic dispenser unit. This is often due to the fact that the ventilator and the anesthetic dispenser unit are kept in a relatively cool room until they are used. During the warm-up phase, the temperature of the ventilator and the temperature of the or each inserted anesthetic dispenser unit rises. Therefore, the saturation concentration increases during the warm-up phase.

The control unit is configured as follows: In order to derive the value for the upper concentration threshold, the control unit applies this functional relationship to the measured value of the ambient temperature and also to the measured value of the insertion time span.

According to the invention, the control unit is configured to calculate a value for the upper concentration threshold. In one embodiment, the ventilation system and the control arrangement are configured as follows, and the control process and the ventilation process comprise the following steps: The or each actual temperature sensor of the temperature sensor group repeatedly measures the respective temperature, for example at a respective predetermined sampling rate. The control unit repeatedly calculates a value for the upper concentration threshold and uses the most recent temperature values measured, for example the N most recent values, where N is a predetermined number, or the measured temperature values from a sliding time window. In the warm-up phase described above, the calculated value for the upper concentration threshold is generally always greater because the ambient temperature is greater than that of the ventilation arrangement and that of the anesthetic dispenser unit. This configuration makes it possible to increase the anesthetic concentration during the warm-up phase without any great risk of the anesthetic condensing.

In a preferred embodiment, the ventilation arrangement comprises an anesthetic sensor. The anesthetic sensor is configured to measure the concentration of the anesthetic in the generated anesthetic gas mixture. The anesthetic sensor is preferably located between the or each receptacle on the one hand and the patient-side coupling unit on the other and is preferably arranged inside the housing of the ventilator. The control unit preferably performs closed-loop control. An objective of this control is to ensure that the measured actual concentration of the anesthetic in the anesthetic gas mixture or in the breathable gas mixture is equal to the calculated target concentration. For this control, the control unit receives and processes a signal from the anesthetic sensor. If there is a large control deviation, i.e. a large deviation between the actual concentration and the target concentration, the control unit controls the anesthetic dispenser unit with the objective of reducing the control deviation.

The invention reduces the risk of anesthetic condensing within the anesthetic sensor. Condensed anesthetic can lead to an incorrect measurement result of the anesthetic sensor and sometimes to damage to a component of the anesthetic sensor.

The temperature sensor on the ventilator side is configured to measure the temperature at the ventilator measuring position. In one embodiment, the ventilator measuring position is in thermal contact with the anesthetic sensor. This embodiment further reduces the risk of anesthetic condensing in the anesthetic sensor.

In the embodiment described so far, the term “the anesthetic” has been used. It is possible that different anesthetics are used. In particular, at least one of the following applications is often possible:
A ventilation arrangement according to the invention is configured to supply a patient with an anesthetic gas mixture containing two different anesthetics. These two anesthetics generally come from two different anesthetic dispenser units, which are inserted simultaneously or at least overlapping in time into two different receptacles in the housing of the ventilator.
Or the same ventilation arrangement according to the invention is configured to supply a first patient with a first anesthetic gas mixture and subsequently a second patient with a second anesthetic gas mixture, wherein the first anesthetic gas mixture comprises a first anesthetic and the second anesthetic gas mixture comprises a second anesthetic which is different from the first anesthetic. For example, a first anesthetic dispenser unit with the first anesthetic and subsequently a second anesthetic dispenser unit with the second anesthetic are inserted and used in the same receptacle.
Or a first ventilation arrangement according to the invention ventilates a patient with a first anesthetic gas mixture, and a second ventilation arrangement according to the invention ventilates the same or another patient with a second anesthetic gas mixture, these two anesthetic gas mixtures in turn comprising different anesthetics and/or different anesthetic concentrations.

As already explained, the saturation concentration of an anesthetic depends on the temperature of a gas mixture comprising the anesthetic and also differs from anesthetic to anesthetic at the same temperature. In one embodiment, the functional relationship for the upper concentration threshold is specified in such a way that its use according to the invention for each anesthetic under consideration prevents the anesthetic from condensing with a high degree of certainty. For example, the anesthetic with the lowest saturation concentration is used to establish the functional relationship. The alternative embodiment described below distinguishes between different anesthetics and therefore makes it possible in many cases to use a higher target concentration for at least one anesthetic than for another, which increases the possible uses of the ventilation arrangement compared to using the lowest saturation concentration.

According to this alternative embodiment, a set with at least two anesthetics to be considered is specified, i.e. a list with these anesthetics. For each anesthetic of the anesthetic set, a respective individual functional relationship is specified in a form that can be evaluated by a computer. This individual functional relationship applies to this anesthetic and specifies the upper concentration threshold depending on the or each measurable temperature. As the measurable temperature increases, the upper concentration threshold specified in the individual functional relationship increases or at least remains the same, as described above for the one functional relationship. The individual correlations may differ from one anesthetic to another.

According to the alternative embodiment, the control unit is configured to calculate a respective target concentration for each anesthetic in question. For this purpose, the control unit captures a specification or a measurement of which anesthetic is contained in the anesthetic gas mixture which the anesthetic dispenser unit is to generate, and applies the individual functional relationship for this anesthetic to the respective measured value of the or each measurable temperature. The application provides a value for the upper concentration threshold that is below the saturation limit of that anesthetic.

This configuration can be implemented in combination with an application in which the ventilation arrangement actually only uses a single anesthetic. Preferably, it is then specified or measured which anesthetic this is, and the control unit always applies the individual functional relationship for this one specified anesthetic. However, because several individual relationships are predefined and stored, a ventilation arrangement according to the invention can still be used later for a different anesthetic. This increases flexibility.

In a preferred implementation of the embodiment with the multiple predetermined individual relationships, the control unit is configured to capture, for the receptacle or for each receptacle in the housing, which anesthetic the anesthetic gas mixture comprises, which anesthetic gas mixture is generated and provided by an anesthetic dispenser unit in this receptacle. For example, the control unit captures an input from a user. Or the ventilation arrangement comprises a reader for each receptacle, wherein the reader is configured to read an identification on a surface or in a data memory of the anesthetic dispenser unit inserted in this receptacle. The identification can, for example, be stored on an NFC chip, in particular an RFID chip, or comprise a bar code (bar pattern) or a QR code or a sequence of alphanumeric characters or a color code. The control unit evaluates a signal from the reader and therefore “knows” which anesthetic the anesthetic gas mixture generated by the anesthetic dispenser unit in this receptacle contains. The control unit selects the individual functional relationship for the anesthetic captured and applies this to the measured value of each measurable temperature.

According to the invention, the control unit is configured to calculate a target concentration for the concentration of the anesthetic in the anesthetic gas mixture. In one embodiment, the control unit is configured to capture a specification. The captured specification specifies a concentration or a volume flow or a mass flow of the anesthetic in the anesthetic gas mixture to be generated. The specification can originate from a user or from a higher-level control system. In particular, the specification can specify what concentration the anesthetic should have in the breathable gas mixture that reaches the patient-side coupling unit. The control unit is configured to derive the target concentration depending on the captured specification and thereby calculate it. In the simplest case, the control unit uses the captured specification as the target concentration. According to this embodiment, the control process and the ventilation process comprise the corresponding steps.

In one embodiment, the ventilation system and the control arrangement comprise an input unit. With the aid of this input unit, a user is able to make the specification just described for a concentration or a volume flow or a mass flow of the anesthetic, and the input unit captures the user input. Preferably, the input unit is configured to offer a range of values to a user. The user can make the setting by selecting a value from the offered range of values, for example using a slider. The input unit is configured to capture this selection, and the captured selection is transmitted to the control unit.

Preferably, the control unit is configured to calculate in advance an upper threshold for this value range. To calculate this upper threshold, the control unit uses the value derived according to the invention for the upper concentration threshold. The control unit calculates the upper threshold for the value range as follows: Each value from the range of values leads to a target concentration of the anesthetic in the anesthetic gas mixture that is at most equal to the value for the upper concentration threshold. This prevents the following undesirable event: The user selects a value from the range of values, but this value would lead to an anesthetic concentration that is too high, i.e. to an anesthetic concentration at which there is a relatively high risk of anesthetic condensation. In particular, the invention avoids the need to deviate from a user input or to issue a message to the user according to which the user input cannot be realized.

Preferably, the ventilation arrangement provides a ventilation circuit. The gas mixture exhaled by the patient flows from the patient-side coupling unit back to the ventilator. Because the breathable gas mixture contains at least one anesthetic, the exhaled gas mixture usually also contains this anesthetic. Because a ventilation circuit is provided, the risk of this anesthetic getting into an environment of the ventilation arrangement is reduced.

In the following, the invention is described by means of exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic sectional view of the ventilation arrangement of an exemplary embodiment;

FIG. 2 is a schematic sectional view of the ventilation arrangement shown in FIG. 1;

FIG. 3 is a schematic sectional view of a ventilation arrangement with two anesthetic dispenser units;

FIG. 4 is a schematic view showing how the control unit calculates the upper concentration threshold when three temperature sensors are present; and

FIG. 5 is a schematic view showing how the control unit calculates the upper concentration threshold when only one sensor for the ambient temperature is present.

### DESCRIPTION OF PREFERRED EMBODIMENTS

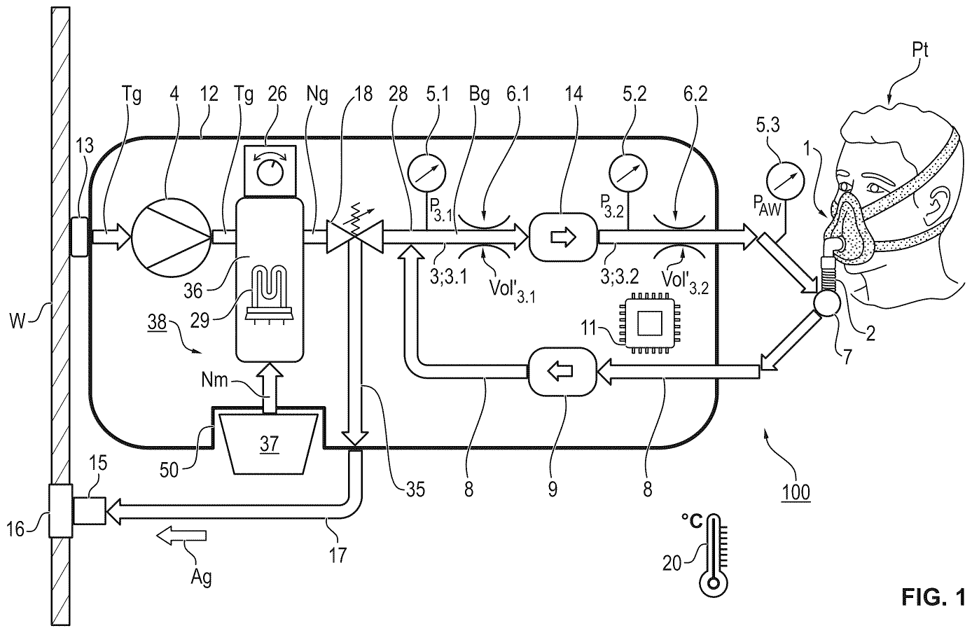

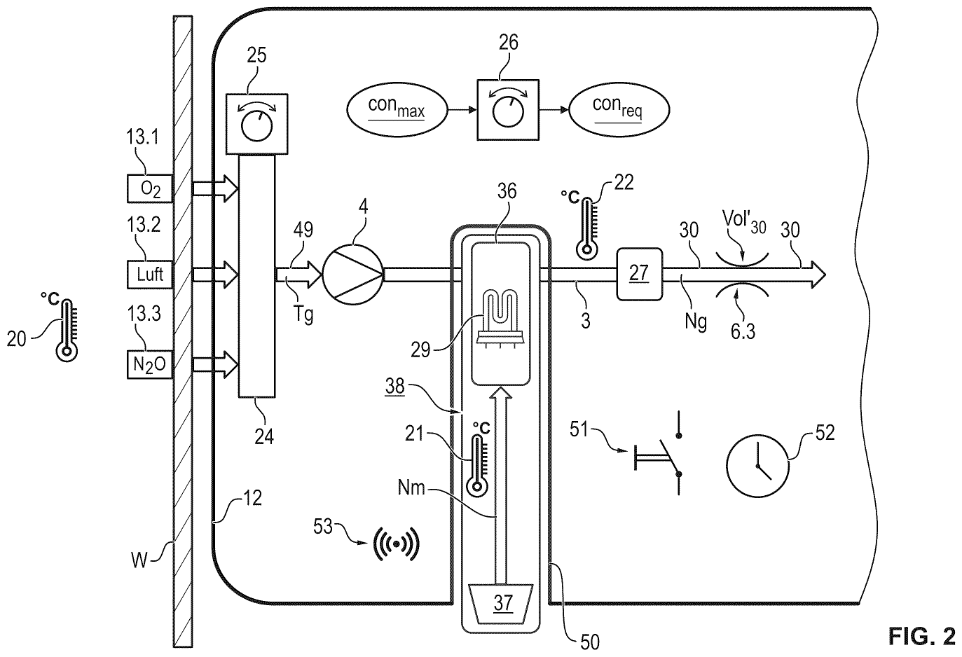

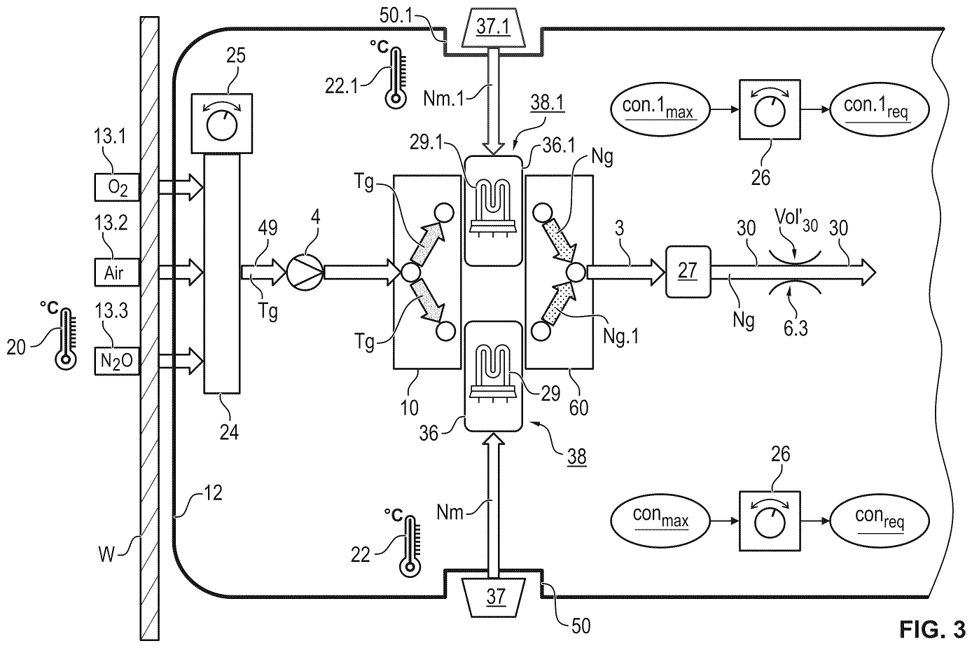

Referring to the drawings, FIG. 1 schematically shows a preferred application of the invention in a ventilation arrangement that provides a ventilation circuit. FIG. 2 schematically shows a section of the ventilation arrangement of FIG. 1. FIG. 3 schematically shows a section of a ventilation arrangement with two anesthetic dispenser units. Identical reference signs have the same meaning. These three figures also show components of a control arrangement. The ventilation system of the exemplary embodiment comprises the ventilation arrangement described below, and the control arrangement shown in part and described further below.

A ventilation arrangement artificially ventilates a patient Pt. A patient-side coupling unit is attached to and/or in the body of the patient Pt, in the exemplary embodiment a breathing mask 1 on his/her face, optionally a tube in the body of the patient Pt. An inspiratory fluid guide unit, for example a hose, comprises two segments 3.1 and 3.2 described below and connects a schematically shown ventilator 100 to the two legs of a Y-piece 7. The patient-side coupling unit 1 is in fluid connection with the base part of the Y-piece 7 via a patient-side fluid guide unit 2.

In the exemplary embodiment, the patient Pt is anesthetized or at least sedated with the aid of an anesthetic. The ventilator 100 comprises a housing 12 and is connected to a schematically shown supply connection arrangement 13 for breathing air and oxygen and optionally for compressed air and/or for at least one component of a carrier gas for anesthetics. The supply connection arrangement 13 is embedded in a wall W or in a supply unit, for example in a ceiling supply unit. Part of the inspiratory fluid guide unit is located inside the housing 12, the remaining part outside the housing 12.

In the exemplary embodiment, the supply connection arrangement 13 is embedded into the wall W. In this example, the supply connection arrangement 13 comprises three individual supply connections, namely a supply connection 13.1 for pure oxygen (O₂), a supply connection 13.2 for breathing air (Air) and a supply connection 13.3 for nitrous oxide (N₂O), see FIG. 2 and FIG. 3. The three gases from the three supply connections 13.1, 13.2, 13.3 are fed to a controllable carrier gas mixer 24. With the aid of a control unit 25, a user can specify the mixing ratio in which the three gases oxygen, breathing air and nitrous oxide are to be mixed to form the carrier gas.

The carrier gas mixer 24 generates a carrier gas Tg according to the control, which has at least one of the three possible components pure oxygen, breathing air and nitrous oxide. A carrier gas fluid guide unit 49 guides the carrier gas Tg to an anesthetic dispenser 36. The anesthetic dispenser 36 receives the carrier gas Tg from the carrier gas mixer 24 on the one hand and a liquid anesthetic Nm from an anesthetic container 37 on the other.

In one embodiment, the anesthetic container 37 is inserted into a receptacle 50 in the housing 12, see FIG. 1 and FIG. 3. In the exemplary embodiment, the anesthetic container 37 can be removed from the receptacle 50 and reinserted into the receptacle 50. This allows, for example, a used anesthetic container 37 to be replaced by a new anesthetic container, wherein the new anesthetic container may contain the same or a different anesthetic.

In another embodiment, the anesthetic dispenser 36 and the anesthetic container 37 belong to an anesthetic dispenser unit 38 (vapor unit). The anesthetic dispenser unit 38 can be removed as a whole from the receptacle 50, and the same or a different anesthetic dispenser unit 38 can be reinserted into the receptacle 50, see FIG. 2. The anesthetic dispenser unit 38 comprises at least two pneumatic coupling points. Two corresponding coupling points are inserted into the receptacle 50. When the anesthetic dispenser unit 38 is inserted, the carrier gas Tg flows from the ventilator 100 into the anesthetic dispenser unit 38 on the one hand. On the other hand, an anesthetic gas mixture Ng flows from the anesthetic dispenser unit 38 into the ventilator 100. Preferably, the ventilator 100 also supplies the anesthetic dispenser unit 38 with electrical energy by means of two corresponding electrical interfaces.

In the following, the term “the anesthetic dispenser unit 38” is used for short, and this can refer to both the implementation with the anesthetic container 37 as the anesthetic dispenser unit 38 or the anesthetic dispenser 36 and the anesthetic container 37 as the anesthetic dispenser unit 38 according to FIG. 1 and FIG. 3 as well as the implementation with the anesthetic dispenser 36 and the anesthetic container 37 as the anesthetic dispenser unit 38 according to FIG. 2.

A contact switch 51 detects whether an anesthetic dispenser unit 38 is inserted into the receptacle 50 or not. The position of the contact switch 51 shown in FIG. 2 is only an example. A system clock 52 measures the time period that has elapsed since an anesthetic dispenser unit 38 was inserted into the receptacle 50 and has not yet been removed again (insertion time span). A reader 53 reads a marking on an anesthetic dispenser unit 38 that is inserted into the receptacle 50, preferably without contact. This marking specifies which anesthetic contains the anesthetic gas mixture Ng, which is generated and provided by the inserted anesthetic dispenser unit 38.

In the embodiment shown in FIG. 3, two receptacles 50, 50.1, each for one anesthetic container 37, 37.1, are recessed into the housing 12. The anesthetic container 37 belongs to the anesthetic dispenser unit 38, the further anesthetic container 37.1 belongs to a further anesthetic dispenser unit 38.1. The anesthetic dispenser unit 38 also comprises the anesthetic dispenser 36, the further anesthetic dispenser unit 38.1 comprises a further anesthetic dispenser 36.1. The further anesthetic container 37.1 contains a further anesthetic Nm.1. This can be the same as the anesthetic Nm in the anesthetic container 37 or a different anesthetic. A further contact switch and a further reader are arranged on the further receptacle 50.1 (not shown), wherein the further contact switch detects the event that a further anesthetic dispenser unit 38.1 is inserted into the receptacle 50.1, and wherein the further reader reads an identification for the anesthetic Nm.1 of the inserted further anesthetic dispenser unit 38.1. The system clock 52 is also configured to measure the time period that has elapsed since the further anesthetic dispenser unit 38.1 has been inserted into the further receptacle 50.1.

The anesthetic dispenser 36, 36.1 evaporates or vaporizes liquid anesthetic from the anesthetic container 37, 37.1 in a feed chamber (not shown) and feeds at least one gaseous anesthetic Nm, Nm.1 into a stream of the carrier gas Tg. A heater 29 is shown schematically in the feed chamber of the anesthetic dispenser 36. A further heater 29.1 is arranged in the feed chamber of the further anesthetic dispenser 36.1. The further heater 29, 29.1 contributes to vaporizing or otherwise evaporating a liquid anesthetic Nm, Nm.1 that is fed into the feed chamber. In an alternative implementation, the anesthetic dispenser 36, 36.1 injects the liquid anesthetic Nm, Nm.1 into the feed chamber, and the injected anesthetic Nm, Nm.1 is evaporated, so that a saturated vapor with an anesthetic concentration close to the saturation concentration is generated. This saturated vapor is mixed with the carrier gas stream. The injection can be continuous or pulsed.

By feeding, the anesthetic dispenser 36 generates a mixture of the carrier gas Tg and the anesthetic Nm from the anesthetic container 37. Accordingly, the further anesthetic dispenser 36.1 generates a mixture of the same carrier gas Tg and the same or a different anesthetic Nm.1 from the anesthetic container 37.1. This mixture is hereinafter referred to as the anesthetic gas mixture Ng or Ng.1. The anesthetic gas mixture Ng.1 may contain anesthetics with a different concentration and/or a different anesthetic than the anesthetic gas mixture Ng.

In the exemplary embodiment, a user can specify a target concentration con_{req} of the anesthetic Nm in the anesthetic gas mixture Ng using a control unit 26. Here, the user selects a value from a predefined value range. An upper concentration threshold conₘₐₓ for the target concentration con_{req} limits this value range upwards. The user can therefore specify at most the value conₘₐₓ for the upper concentration threshold Conₘₐₓ as the target concentration con_{req}. The same applies to the target concentration con.1_{req} of the further anesthetic Nm.1 in the anesthetic gas mixture Ng.1 from the further anesthetic dispenser unit 38.1: The target concentration con.1_{req} is at most the same as a calculated value con.1ₘₐₓ. The two target concentrations con_{req} and con.1_{req} can preferably be determined independently of each other.

In an alternative embodiment, not shown, a user can use a corresponding control unit (not shown) to specify a target concentration of the anesthetic Nm in the ventilation gas mixture Bg, preferably as the target concentration at the Y-piece 7 and thus in the patient-side coupling unit 1. The control unit 11 derives the target concentration con_{req} of the anesthetic Nm in the anesthetic gas mixture Ng from this specification and from other specifications and/or measured values. Again, the target concentration con_{req} is at most as high as the upper concentration threshold conₘₐₓ.

FIG. 2 shows schematically that a target concentration con_{req} results from a user specification by means of the control unit 26. FIG. 3 also shows schematically that a target concentration con.1_{req} results from a user specification. A signal-processing control unit 11 uses this target concentration con_{req} to control the inserted anesthetic dispenser unit 38. The objective of this control is to ensure that the actual concentration of the anesthetic Nm in the anesthetic gas mixture Ng is equal to the target concentration con_{req}. Accordingly, a user specification results in a target concentration con.1_{req} for the anesthetic Nm.1 in the further anesthetic gas mixture Ng.1. During control, for example, a volume flow or mass flow of the carrier gas Tg and/or the thermal energy emitted by the heater 29, 29.1 of the anesthetic dispenser unit 38, 38.1 is changed. Or a volume flow or mass flow or a pulse rate at which anesthetic is injected into the feed chamber is changed.

In the embodiment shown in FIG. 3, two anesthetic dispenser units 38 and 38.1 are simultaneously inserted into the two receptacles 50 and 50.1. Preferably, the user selects a target concentration from a value range for each anesthetic dispenser unit 38, 38.1, i.e. a total of two target concentrations con_{req}, con.1_{req} from two value ranges. Each value range is limited at the top by a value [Conₘₐₓ, Con.1ₘₐₓ] for an upper concentration threshold Conₘₐₓ, Con.1ₘₐₓ. These two values can differ from each other.

In the embodiment shown in FIG. 3, an optional pneumatic switching valve 10 directs the carrier gas Tg, depending on the position of the switching valve 10, either to the anesthetic dispenser unit 38 and thus to the anesthetic dispenser 36 or to the further anesthetic dispenser unit 38.1, and thus to the further anesthetic dispenser 36.1. A merging unit 60 feeds the anesthetic gas mixture Ng from the anesthetic dispenser unit 38 and the further anesthetic gas mixture Ng.1 from the further anesthetic dispenser unit 38.1 into a supply fluid guide unit 30.

In an alternative embodiment, the carrier gas Tg is conducted at least temporarily to both anesthetic dispenser units 38, 38.1 and thus to both inserted anesthetic dispensers 36, 36.1. According to this alternative embodiment, the component 10 divides the carrier gas Tg between the two anesthetic dispenser units 38, 38.1. The patient Pt is therefore supplied with a mixture of both anesthetic gas mixtures Ng, Ng.1.

An anesthetic sensor 27 measures an indicator of the actual concentration of anesthetic in the anesthetic gas mixture Ng, Ng.1 flowing through the supply fluid guide unit 30. In the exemplary embodiment, the anesthetic sensor 27 is located downstream of the anesthetic dispenser unit or each anesthetic dispenser unit 38, 38.1 and upstream of the feed point 28.

In one embodiment, the control unit 11 uses a signal from the anesthetic sensor 27 to regulate the concentration of anesthetic Nm, Nm.1 in the anesthetic gas mixture Ng, Ng.1 and thus in the ventilation gas mixture Bg flowing to the patient-side coupling unit 1. A time course or a value for the target concentration con_{req}, con.1_{req} of the anesthetic Nm, Nm.1 is specified. The control unit 11 controls the anesthetic dispenser 36, 36.1 or a valve with the control objective that the actual anesthetic concentration equals or follows the target concentration con_{req}, con.1_{req}.

In the exemplary embodiment, a third volumetric flow sensor 6.3 is arranged in the supply fluid guide unit 30. The third volume flow sensor 6.3 measures the volume flow Vol′30 through the feed fluid guide unit 30.

The control unit 11 or a separate evaluation unit uses a signal from the anesthetic sensor 27 and a signal from the third volume flow sensor 6.3 to derive the quantity of anesthetic that has flowed through the supply fluid guide unit 30 in a certain time period. The third volume flow sensor 6.3 is also arranged between the anesthetic dispenser unit 38, 38.1 and the feed point 28. The two sensors 27 and 6.3 are connected in series. As shown in FIG. 2 and FIG. 3, the volume flow sensor 6.3 can be arranged downstream of the anesthetic sensor 27 or also upstream of the anesthetic sensor 27.

The supply fluid guide unit 30 guides the anesthetic gas mixture Ng, Ng.1 to a feed point 28, see FIG. 1. The anesthetic gas mixture Ng, Ng.1 is fed into a ventilation circuit at the feed point 28.

The ventilator 100 ejects a breathable gas mixture comprising oxygen and at least one anesthetic Nm, Nm.1. This gas mixture is referred to as the ventilation gas mixture Bg and comprises the anesthetic gas mixture Ng, Ng.1. Preferably, the ventilator 100 performs a sequence of ventilation strokes and ejects a quantity of the ventilation gas mixture Bg from the housing 12 in each ventilation stroke. The expelled ventilation gas mixture Bg flows through the inspiratory fluid guide unit 3.1, 3.2 to the Y-piece 7 and further through the patient-side fluid guide unit 2 and is inhaled by the patient Pt with the aid of the patient-side coupling unit 1.

A fluid conveying unit, for example a blower 4 or a pump or a piston-cylinder unit, generates a volume flow, for example a constant volume flow over time, and a pressure, for example a constant pressure over time. The constant pressure over time is between 10 mbar and 100 mbar, for example. The position of the fluid guide unit 4 shown is only an example.

A first pressure sensor 5.1 measures the actual pressure P_{3.1} in the first segment 3.1. A second pressure sensor 5.2 measures the actual pressure P_{3.2} in the second segment 3.2. A third pressure sensor 5.3 measures the pressure in the patient-side fluid guide unit 2 and thus the pressure in the airway (pressure in airway, PAW). A first volume flow sensor 6.1 measures the actual volume flow Vol′_{3.1} through the first segment 3.1. A second volume flow sensor 6.2 measures the actual volume flow Vol′_{3.2} through the second segment 3.2. To be more precise: each sensor 5.1, 5.2 measures a variable that correlates with the actual pressure. Each sensor 6.1, 6.2, 6.3 measures a variable that correlates with the actual volume flow. Of course, not all of these sensors necessarily need to be present.

The control unit 11 receives a signal from each of the sensors 5.1, 5.2, 5.3 and 6.1, 6.2 and controls a valve arrangement 14 with at least one valve. The valve arrangement 14 is located between the first segment 3.1 and the second segment 3.2. In one embodiment, the control unit 11 performs closed-loop control with the control objective that the actual time course of the volume flow Vol′3.2 to the patient-side coupling unit 1 or the pressure PAW at the patient-side coupling unit 1 follows a predetermined target time course. Another or additional possible control objective is the following: The quantity of the ventilation gas mixture Bg flowing to the patient-side coupling unit 1 during an inspiration phase should be equal to a predetermined target quantity, with a determined tidal volume of the patient's lung Pt, for example, being specified as the target quantity.

An expiratory fluid guide unit 8, for example another hose, leads from the Y-piece 7 back to the ventilator 100—more precisely: back to the feed point 28. The gas mixture that the patient Pt has exhaled flows through the expiratory fluid guide unit 8. An end-expiratory valve 9 is preferably arranged in the expiratory fluid guide unit 8, which ensures that a minimum pressure is maintained in the lungs of the patient Pt. Preferably, a CO2 absorber (not shown) in the expiratory fluid guide unit 8 removes carbon dioxide from the exhaled gas mixture.

As a rule, the gas mixture exhaled by the patient Pt contains anesthetic. This anesthetic should not be released into the environment. Therefore, a ventilation circuit is implemented between the ventilator 100 and the patient-side coupling unit 1. The gas mixture exhaled by the patient Pt is fed back into the flow of the gas mixture, which the fluid conveying unit 4 keeps moving, thanks to the ventilation circuit.

The anesthetic gas mixture Ng, Ng.1 is fed into this ventilation circuit at the feed point 28. The mixture of the injected anesthetic gas mixture Ng, Ng.1 and the exhaled gas mixture, which is returned through the expiratory fluid guide unit 8, form the ventilation gas mixture Bg of the exemplary embodiment.

An excess gas mixture (exhaust gas Ag) must be diverted from this ventilation circuit at least temporarily, see FIG. 1. A pressure relief valve 18 is shown as an example. A fluid guide unit 35 in the ventilator 100 leads from the ventilation circuit or from the pressure relief valve 18 to a connection in the housing 12 of the ventilator 100. A fluid guide unit 17, for example a hose, guides the diverted gas mixture Ag from the ventilator 100 to the wall W. A plug 15 at the free end of the fluid guide unit 17 can be plugged into a socket 16 in the wall W. The diverted gas mixture Ag is fed into the ventilator 100. The diverted gas mixture Ag flows through the fluid guide units 35 and 17, through the plug 15 and the socket 16, into a stationary receiving network behind the wall W, not shown.

In the exemplary embodiment, the ventilation system and the control arrangement comprise all three temperature sensors of the temperature sensor group, namely the following:
An ambient temperature sensor 20 measures the temperature in an area surrounding the ventilator 100.
An anesthetic dispenser temperature sensor 21 on the anesthetic dispenser side is associated with the anesthetic dispenser unit 38 and measures the temperature of the anesthetic dispenser unit 38. The anesthetic dispenser unit 38.1 includes a further anesthetic dispenser temperature sensor on the anesthetic dispenser side, which is not shown.
A receptacle-side temperature sensor 22 operates as the ventilator side temperature sensor measures the temperature at the receptacle 50 for the anesthetic dispenser unit 38. A further receptacle-side temperature sensor 22.1 on the intake side measures the temperature at the receptacle 50.1 for the anesthetic dispenser unit 38.1.

The anesthetic dispenser temperature sensor 21 on the anesthetic dispenser side is in thermal contact with the surface of the anesthetic dispenser unit 38 that faces the receptacle 50 when the anesthetic dispenser unit 38 is inserted. Therefore, the anesthetic dispenser temperature sensor 21 on the anesthetic dispenser side measures the temperature of this surface. In the exemplary embodiment, this surface acts as the anesthetic dispenser measuring position. The same applies to the other anesthetic dispenser temperature sensor on the anesthetic dispenser side of the anesthetic dispenser unit 38.1. Preferably, even in the embodiment in which only the anesthetic container 37, 37.1 can be inserted into the receptacle 50, 50.1, the anesthetic dispenser unit 38, 38.1 comprises the anesthetic dispenser temperature sensor 21, 21.1 on the anesthetic dispenser side.

The receptacle-side temperature sensor 22, 22.1 on the receptacle side functions as the ventilator temperature sensor of the exemplary embodiment and is in thermal contact with a surface of the receptacle 50, 50.1, wherein this surface faces an inserted anesthetic dispenser unit 38, 38.1 and functions as the ventilator measurement position. Preferably, the receptacle-side temperature sensor 22, 22.1 is also in thermal contact with a wall of the supply fluid guide unit 30. Preferably, the receptacle-side temperature sensor 22, 22.1 is arranged between a receptacle-side pneumatic coupling point in the receptacle 50, 50.1 and the anesthetic sensor 27. The anesthetic gas mixture Ng, Ng.1 flows through this coupling point into the ventilator 100. In one embodiment, the anesthetic sensor 27 comprises a base plate made of a metal, and the receptacle-side temperature sensor 22, 22.1 on the receptacle side measures the temperature of the base plate. In internal tests, the inventors have found that the temperature of the base plate deviates only slightly from the temperature of the surface of the receptacle 50, 50.1 facing the anesthetic dispenser unit 38, 38.1.

The inventors have identified the following problem internally: Gaseous anesthetic Nm, Nm.1 in the generated anesthetic gas mixture Ng or Ng.1 can condense and settle on a wall of the ventilation arrangement if the anesthetic Nm, Nm.1 has too high a concentration. This condensation can occur in the anesthetic dispenser unit 38, 38.1 and/or in the ventilator 100. Condensed anesthetic Nm, Nm.1 in the supply fluid guide unit 30 can flow into the anesthetic sensor 27 and falsify a measurement result of the anesthetic sensor 27. In particular, condensed anesthetic Nm, Nm.1 can cause the anesthetic sensor 27 to measure an anesthetic concentration that is too low and therefore the patient Pt is supplied with too much anesthetic Nm, Nm.1. The condensation of anesthetic Nm, Nm.1 can also lead to the patient Pt receiving too little anesthetic Nm, Nm.1. The invention significantly reduces the risk of gaseous anesthetic Nm, Nm.1 condensing.

It has already been explained above that a user can use the control unit 26 to specify a target concentration con_{req}, con.1_{req} of the anesthetic Nm or Nm.1 in the generated anesthetic gas mixture Ng or Ng.1. This target concentration con_{req}, con.1_{req} lies within a value range that is limited at the top by an upper concentration threshold conₘₐₓ or con.1ₘₐₓ. In the exemplary embodiment, the user can use the control unit 26 to specify a target concentration con_{req}, con.1_{req} for each of the two anesthetic dispenser units 38 and 38.1, i.e. a total of two target concentrations con_{req}, con.1_{req}, wherein these two target concentrations con_{req}, con.1_{req} can differ from one another.

A conceivable remedy for the above-mentioned problem that anesthetic Nm, Nm.1 can condense would be the following: The upper concentration threshold conₘₐₓ, con.1ₘₐₓ is specified so small that anesthetic Nm, Nm.1 cannot condense in any situation. However, this would limit the possible uses of the ventilator 100. Another conceivable remedy would be to heat the entire supply fluid guide unit 30 or at least one segment. The invention can be used in combination with such a heater. However, the invention presents a different or additional way to at least largely prevent anesthetic from condensing.

The inventors have internally identified the following possible cause of condensation: Frequently, an anesthetic dispenser unit 38, 38.1 is kept in stock in a storage room or other storage area and is retrieved from this storage area when needed and inserted into a receptacle 50, 50.1 of the ventilator 100. In particular to prevent anesthetic Nm, Nm.1 from already evaporating in the anesthetic dispenser unit 38, 38.1, the storage area has a relatively low temperature, in particular a lower temperature than the room in which the patient Pt is supplied with the anesthetic gas mixture Ng or Ng.1. In addition, the ventilator 100 is often kept in a cooler storage room. If the anesthetic dispenser unit 38, 38.1 is later inserted into the receptacle 50, 50.1 and the anesthetic gas mixture Ng or Ng.1 is then generated, the anesthetic dispenser unit 38, 38.1 and optionally also the receptacle 50, 50.1 initially still have the temperature of the storage area and only gradually warm up, namely in a warm-up phase, to the ambient temperature in the vicinity of the ventilator 100 used.

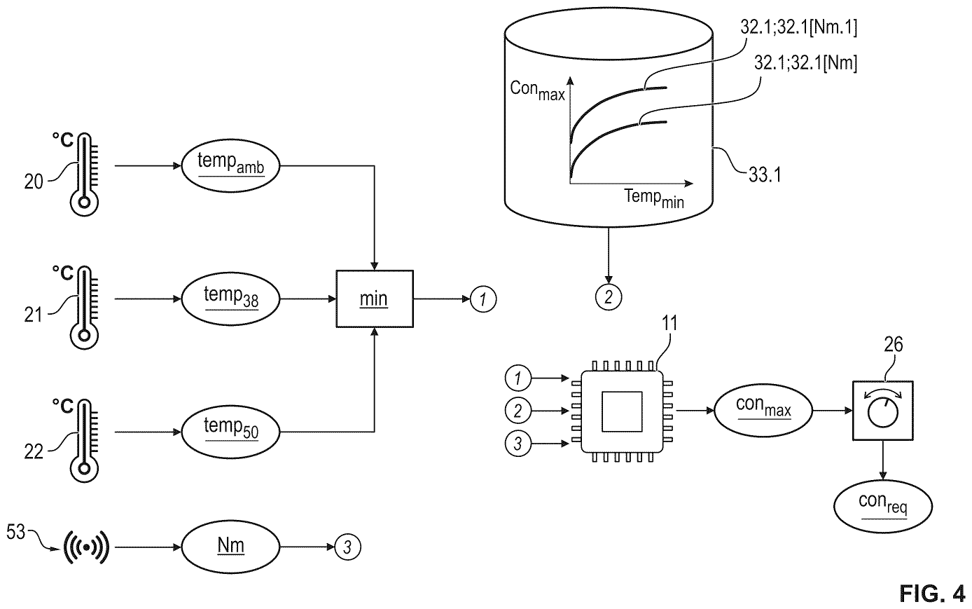

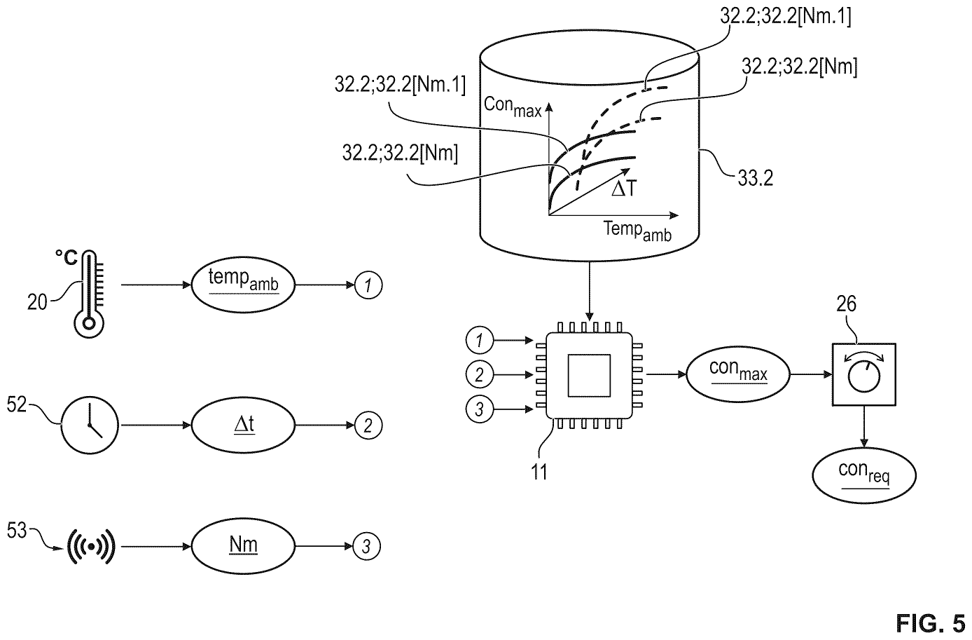

According to the invention, the control unit 11 calculates the upper concentration threshold conₘₐₓ, con.1ₘₐₓ for the range of values from which the user can select a desired target concentration con_{req}, con.1_{req} with the aid of the control unit 26 and specify it to the anesthetic dispenser unit 38, 38.1. FIG. 4 and FIG. 5 schematically illustrate two embodiments of how the control unit 11 performs this. In the embodiment shown in FIG. 4, the three temperature sensors 20, 21, 22 are used. In the configuration shown in FIG. 5, only the ambient temperature sensor 20 is used. Of course, it is also possible to use three temperature sensors 20, 21, 22 in the configuration shown in FIG. 5.

Note: In this representation, the name of a physical quantity is indicated with an upper-case letter at the beginning, the name of a measured value of this physical quantity with a lower-case letter.

The ambient temperature sensor 20 provides a measured value temp_{amb} for the ambient temperature Temp_{amb}. The anesthetic dispenser temperature sensor 21 on the anesthetic dispenser side provides a measured value temp₃₈ for the temperature Temp₃₈ on the surface of the anesthetic dispenser unit 38 facing the receptacle 50. The receptacle-side temperature sensor 22 on the receptacle side provides a measured value temp₅₀ for the measurable temperature Temp₅₀ on the surface of the receptacle 50 facing the anesthetic dispenser unit 38. Preferably, the three temperature sensors 20, 21, 22 measure the respective temperature repeatedly, for example at a fixed sampling rate. The system clock 52 provides a measured value ΔT for the time period Δt that has elapsed since the anesthetic dispenser unit 38 was inserted into the receptacle 50. A reader 53 captures an identifier on a surface or in a data memory of the anesthetic dispenser unit 38. This identifier determines which anesthetic Nm the anesthetic gas mixture Ng provided by the anesthetic contained in the anesthetic dispenser unit 38.

In the embodiment shown in FIG. 4, a functional unit min calculates the minimum of the three measured temperatures temp_{amb}, temp₃₈ and temp₅₀. Another calculation rule for combining the three measured temperatures temp_{amb}, temp₃₈ and temp₅₀ into one value is also possible, for example a weighted average or the mean of the three values.

In the configuration shown in FIG. 4 (three temperature sensors 20, 21, 22), a functional relationship 32.1 is specified in a form that can be evaluated by a computer and is stored in a data storage 33.1. This functional relationship 32.1 describes the upper concentration threshold Conₘₐₓ as a function of the temperature minimum Tempₘᵢₙ=min (Temp_{amb}, Temp₃₈, Temp₅₀). The functional relationship 32.1 is configured in such a way that the upper concentration threshold Conₘₐₓ is the greater the greater the temperature minimum Tempₘᵢₙ is.

This functional relationship 32.1 is established before the ventilation arrangement is used. In the exemplary embodiment, the functional relationship 32.1 comprises an individual functional relationship 32.1 [Nm], 32 [Nm.1] for each anesthetic Nm, Nm.1 under consideration. Each individual functional relationship 32.1 [Nm], 32 [Nm.1] for an anesthetic Nm, Nm.1 is configured in such a way that the upper concentration threshold Conₘₐₓ for this anesthetic Nm, Nm.1 is greater the greater the temperature minimum Tempₘᵢₙ is.

The physical background for establishing the individual functional relationships is as follows: As mentioned above, every anesthetic has a saturation concentration. The higher the temperature of a gas mixture containing this anesthetic, the higher the saturation concentration for this anesthetic. Exemplary values are:

| | Saturation concentration | Saturation concentration |
|---|---|---|
| Anesthetic | at 10° C. in [Vol.-%] | at 20° C. in [Vol.-%] |
| Isoflurane | 19 | 31 |
| Sevoflurane | 12 | 20 |
| Desflurane | 58 | 88 |

These values are known and are specified. A safety margin is also specified. Use is made of the fact that the temperature of the anesthetic gas mixture Ng, Ng.1 with the anesthetic Nm, Nm.1 is at least as high as the temperature minimum Tempₘᵢₙ minus the predetermined safety margin. A calibration device generates the individual functional relationships 32.1 [Nm], 32 [Nm.1] in advance using the predetermined saturation concentrations and the safety margin.

The control unit 11 applies the functional relationship 32.1 to the calculated value tempₘᵢₙ for the temperature minimum Tempₘᵢₙ, which is provided by the functional unit min. In the example shown in FIG. 4 and FIG. 5, the control unit 11 calculates a value conₘₐₓ for the upper concentration threshold Conₘₐₓ, wherein the value conₘₐₓ refers to the anesthetic dispenser unit 38 inserted in the receptacle 50 and conveying the anesthetic gas mixture Ng with the anesthetic Nm. The value con.1ₘₐₓ is calculated in the same way.

The control unit 11 receives the information from the reader 53 that the inserted anesthetic dispenser unit 38 is configured to generate and supply an anesthetic gas mixture Ng with the anesthetic Nm. The control unit 11 selects the individual functional relationship 32.1 [Nm] for this anesthetic Nm. The control unit 11 applies the selected individual functional relationship 32.1 [Nm] to the measured value temp_{amb} of the ambient temperature Temp_{amb}. Through the application, the control unit 11 generates a value conₘₐₓ for the upper concentration threshold Conₘₐₓ.

This value conₘₐₓ for the upper concentration threshold Conₘₐₓ is transmitted to the control unit 26. Preferably, the control unit 26 is configured in such a way that the user is only offered the value range from zero to the transmitted value conₘₐₓ of the upper concentration threshold Conₘₐₓ for selection. This avoids the event that the user selects a value con_{req} for the desired anesthetic concentration, but this value con_{req} is not realized because it is greater than the transmitted value conₘₐₓ for the upper concentration threshold Conₘₐₓ.

Preferably, each temperature sensor 20, 21, 22 repeatedly measures the respective temperature Temp_{amb}, Temp₃₈, Temp₅₀. As a rule, the ambient temperature Temp_{amb} remains approximately constant. On the other hand, the measured temperatures Temp₃₈ and Temp₅₀ often increase because the temperature Temp₅₀ of the ventilator 100 and the temperature Temp₃₈ of the anesthetic dispenser unit 38 adapt to the ambient temperature Temp_{amb}. The application of the functional relationship 38.1 or the selected individual functional relationship simulates this increase. One effect is as follows: At the beginning of an application, the value of the upper concentration threshold conₘₐₓ is smaller than after a longer insertion time span. The process of the temperature Temp₅₀ of the ventilator 100 and the temperature Temp₃₈ of the anesthetic dispenser unit 38 adapting to the ambient temperature Temp_{amb} means that the anesthetic concentration can be increased without a major risk of condensation occurring.

In the embodiment according to FIG. 5, a functional relationship 32.2 is predefined in computer-evaluable form and is stored in a data storage 33.2. The functional relationship 32.2 comprises a respective individual functional relationship 32.2 [Nm], 32.2 [Nm.1] for each anesthetic Nm, Nm.1. Each individual functional relationship 32.2 [Nm], 32.2 [Nm.1] describes the upper concentration threshold Conₘₐₓ as a function of the measured ambient temperature Temp_{amb} and the time period ΔT that has elapsed since the anesthetic dispenser unit 38 was inserted into the receptacle 50. Each individual functional relationship 32.2 [Nm], 32.2 [Nm.1] is therefore a three-dimensional relationship. Again, for each value Δt of the time span ΔT, the greater the measured ambient temperature Temp_{amb}, the greater the upper concentration threshold Conₘₐₓ. In other words, for a constant time period, the greater the measured ambient temperature Temp_{amb}, the greater the upper concentration threshold Conₘₐₓ. On the other hand, each individual functional relationship 32.2 [Nm], 32.2 [Nm.1] is preferably given as follows: At each value temp_{amb} of the ambient temperature Temp_{amb}, the upper concentration threshold Conₘₐₓ increases in a warm-up phase and then remains constant. This embodiment takes into account the following fact: In the warm-up phase, the temperature Temp₅₀ of the ventilator 100 and thus that of the intake 50 and the temperature Temp₃₈ of the anesthetic dispenser unit 38 adapt to the constant ambient temperature Temp_{amb} and then remain the same.

Again, a calibration device preferably generates each individual functional relationship 32.2 [Nm], 32.2 [Nm.1] in advance. On the one hand, a saturation concentration is predetermined for each anesthetic Nm, Nm.1 and for different temperatures. On the other hand, a lower threshold is preferably specified for the temperature that an anesthetic dispenser unit 38 and a ventilator 100 can have before use. In addition, a sample is empirically generated, the sample comprising a plurality of sample elements. Each sample element is generated as follows: the anesthetic dispenser unit 38 and the ventilator 100—more specifically, the receptacle 50—are each brought to a particular initial temperature. In addition, a certain ambient temperature Temp_{amb} is generated. The anesthetic dispenser unit 38 is inserted into the receptacle 50. The time course of the anesthetic dispenser temperature Temp₃₈ of the inserted anesthetic dispenser unit 38 and the time course of the ventilator temperature Temp₅₀ in the vicinity of the receptacle 50 are measured. As a rule, the time course of the anesthetic dispenser temperature Temp₃₈ of the inserted anesthetic dispenser unit 38 does not depend significantly on which anesthetic Nm, Nm.1 is filled into the anesthetic dispenser unit 38, so that this time course applies to each anesthetic Nm, Nm.1 under consideration. A time course of the saturation concentration is derived for each anesthetic Nm, Nm.1 from the two temperature time courses and from a predetermined safety margin. From this, for each anesthetic Nm, Nm.1 a respective individual functional relationship 32.2 [Nm], 32.2 [Nm.1] is derived and stored.

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | Patient-side coupling unit in the form of a |
| | breathing mask, connected to the fluid guide |
| | unit 2, arranged on the body of the patient Pt |
| 2 | Patient-side fluid guide unit, connects the |
| | Y-piece 7 with the patient-side coupling unit 1 |
| 3.1 | First segment of the inspiratory fluid guide unit, |
| | leads from the fluid conveying unit 4 to the valve |
| | arrangement 14 |
| 3.2 | Second segment of the inspiratory fluid guide unit, |
| | leads from the valve arrangement 14 to the Y-piece 7 |
| 4 | Fluid conveying unit in the form of a blower, |
| | ejects a gas mixture into the first segment 3.1, |
| | is connected to the supply connection 13 |
| 5.1 | First pressure sensor, measures an indicator of |
| | the actual pressure P_{3.1}in the first |
| | segment 3.1, the pressure generally being generated |
| | by the fluid conveying unit 4 |
| 5.2 | Second pressure sensor, measures an indicator of |
| | the actual pressure P_{3.2} in the second segment 3.2 |
| 5.3 | Third pressure sensor, measures an indicator of |
| | the actual pressure in the patient-side coupling |
| | unit 1, usually the airway pressure P_{AW} |
| 6.1 | First volume flow sensor, measures an indicator of |
| | the actual volume flow Vol′_{3.1} through the |
| | first segment 3.1 |
| 6.2 | Second volume flow sensor, measures an indicator |
| | of the actual volume flow Vol′3.2 through the |
| | second segment 3.2 |
| 6.3 | Third volume flow sensor, measures an indicator of |
| | the actual volume flow Vol′₃₀ through the |
| | supply fluid guide unit 30 |
| 7 | Y-piece, connects the fluid guide units 3.2 and 8 |
| | on one side with the patient-side fluid guide unit |
| | 2 on the other side |
| 8 | Expiratory fluid guide unit, leads from the |
| | Y-piece 7 back to the ventilator 100 |
| 9 | End-expiratory valve in the expiratory fluid |
| | guide unit 8 |
| 10 | Pneumatic switching valve, directs the carrier gas Tg from |
| | the carrier gas mixer 24 either to the anesthetic dispenser |
| | unit 38 or to the anesthetic dispenser unit 38.1 or divides |
| | the carrier gas Tg between the two anesthetic dispenser |
| | units 38, 38.1 |
| 11 | Signal-processing control unit, receives and processes |
| | signals from the sensors 5.1, 5.2, 5.3 and 6.1, 6.2, 6.3, |
| | controls the valve arrangement 14 and the carrier gas |
| | mixer 24, calculates the target concentration of the |
| | anesthetic Nm |
| 12 | Housing of the ventilator 100, has the receptacles 50, 50.1 |
| 13 | Supply connection arrangement of the ventilator 100, |
| | connected to the fluid conveying unit 4, comprises the |
| | supply connections 13.1, 13.2, 13.3 for the three possible |
| | components of the carrier gas Tg |
| 13.1 | Supply connection for breathing air, belongs to the |
| | supply connection arrangement 13 |
| 13.2 | Supply connection for nitrous oxide (N₂O), belongs to |
| | supply connection arrangement 13 |
| 13.3 | Supply connection for pure oxygen, belongs to the supply |
| | connection arrangement 13 |
| 14 | Valve arrangement, arranged between the segments 3.1 and |
| | 3.2, comprises at least one controllable valve |
| 15 | Plug at the free end of the fluid guide unit 17, can be |
| | plugged into the socket 16 |
| 16 | Socket in the wall W, accepts the plug 15, connected to a |
| | sink behind the wall W |
| 17 | Fluid guide unit, leads from the fluid guide unit 35 to |
| | the plug 15 |
| 18 | Pressure relief valve in the ventilation circuit, |
| | starting point of the fluid guide unit 35 |
| 20 | Ambient temperature sensor, provides the ambient |
| | temperature Temp_{amb} |
| 21 | Anesthetic dispenser temperature sensor on the |
| | anesthetic dispenser side, provides the |
| | temperature Temp₃₈ |
| 22 | Receptacle-side temperature sensor, functions as |
| | the ventilator-side temperature sensor, provides |
| | the temperature Temp50 at the receptacle 50 |
| | for the anesthetic dispenser unit 38 |
| 22.1 | Further receptacle-side temperature sensor on |
| | the intake (ventilator) side, provides the |
| | temperature at the receptacle 50.1 for the |
| | further anesthetic dispenser unit 38.1 |
| 24 | Carrier gas mixer, generates the carrier gas |
| | Tg from the gases flowing from the supply |
| | connections 13.1, 13.2, 13.3, connected to |
| | the supply connection arrangement 13 |
| 25 | Control unit for the carrier gas mixer 24, allows a user |
| | to set the mixing ratio in the carrier gas Tg |
| 26 | Control unit for the anesthetic dispenser 36, 36.1, allows a |
| | user to specify a target concentration of the anesthetic Nm, |
| | Nm. 1 in the anesthetic gas mixture Ng or Ng.1. |
| 27 | Anesthetic sensor, measures the actual concentration of |
| | anesthetic in the anesthetic gas mixture Ng, Ng. 1 flowing |
| | through the supply fluid guide unit 30 |
| 28 | Feed point at which the anesthetic gas mixture Ng, Ng.1, |
| | which has flowed through the supply fluid guide unit 30, |
| | is fed into the ventilation circuit |
| 29 | Heater in the feed chamber of the anesthetic dispenser 36 |
| 29.1 | Heater in the feed chamber of the additional anesthetic |
| | dispenser 36.1 |
| 30 | Supply fluid guide unit, leads from the anesthetic |
| | dispenser unit 38, 38.1 to the feed point 28 |
| 32.1 | Functional relationship: upper concentration threshold |
| | Conₘₐₓ as a function of temperature |
| 32.1[Nm] | Individual functional relationship for the Nm: upper |
| | concentration threshold Conₘₐₓ as a function |
| | of temperature |
| 32.1[Nm.1] | Individual functional relationship for the Nm.1: |
| | upper concentration threshold Conₘₐₓ as a function |
| | of temperature |
| 32.2 | Functional relationship: upper concentration threshold as a |
| | function of the temperature and the time period ΔT |
| | since the anesthetic dispenser unit 38 was inserted into |
| | the receptacle 50 |
| 32.2[Nm] | Individual functional relationship for the Nm: upper |
| | concentration threshold as a function of the |
| | temperature and the time period ΔT since the anesthetic |
| | dispenser unit 38 was inserted into the receptacle 50 |
| 32.2[Nm.1] | Individual functional relationship for the Nm. 1: |
| | upper concentration threshold as a function of the |
| | temperature and the time period AT since the |
| | anesthetic dispenser unit 38 was inserted into |
| | the receptacle 50 |
| 33.1 | Data storage with the functional relationship 32.1 |
| 33.2 | Data storage with the functional relationship 32.2 |
| 35 | Fluid guide unit in the ventilator 100, connects the |
| | ventilation circuit with the fluid guide unit 17 |
| 36 | Anesthetic dispenser, receives a carrier gas Tg from |
| | the supply connection 13 and liquid anesthetic Nm |
| | from the anesthetic container 37, generates a mixture |
| | Ng of the carrier gas Tg and gaseous anesthetic in a |
| | feed chamber, comprises the heater 29, in one |
| | embodiment belongs to the anesthetic dispenser unit 38 |
| 36.1 | Further anesthetic dispenser, receives a carrier gas |
| | Tg from the supply connection 13 and liquid anesthetic |
| | Nm. 1 from the further anesthetic container 37.1, |
| | generates a mixture Ng.1 of the carrier gas Tg and |
| | gaseous anesthetic in a feed chamber, comprises the |
| | heater 29.1, in one embodiment belongs to the further |
| | anesthetic dispenser unit 38.1 |
| 37 | Container with liquid anesthetic Nm, can be inserted |
| | into the housing 12 and removed from the housing 12 |
| | in one embodiment, belongs to the anesthetic dispenser |
| | unit 38 in another embodiment |
| 37.1 | Additional container with liquid anesthetic Nm. 1 |
| 38 | Anesthetic dispenser unit, comprising the |
| | anesthetic dispenser 36 and the anesthetic container |
| | 37, can be inserted as a whole into the housing 12 |
| | and removed from the housing 12 in one embodiment |
| 38.1 | Further anesthetic dispenser unit, comprising the |
| | anesthetic dispenser 36.1 and the anesthetic |
| | container 37.1 |
| 49 | Carrier gas fluid guide unit, leads from the |
| | carrier gas mixer 24 to the anesthetic dispenser |
| | 36 |
| 50 | Receptacle in the housing 12 for the anesthetic |
| | container 37 or for the anesthetic dispenser |
| | unit 38 |
| 50.1 | Further receptacle in the housing 12 for the further |
| | anesthetic container 37.1 or for the further |
| | anesthetic dispenser unit 38.1 |
| 51 | Contact switch, detects whether or not an anesthetic |
| | dispenser unit 38 is inserted in the receptacle 50 |
| 52 | System clock, measures the time period AT that has |
| | elapsed since the anesthetic dispenser unit 38 was |
| | inserted into the receptacle 50 |
| 53 | Reader on the receptacle 50, reads a marking on an |
| | inserted anesthetic dispenser unit 38, which |
| | identifies the type of anesthetic |
| 60 | Merging unit, feeds the anesthetic gas mixture Ng |
| | from the anesthetic dispenser unit 38 and the |
| | further anesthetic gas mixture Ng. 1 from the |
| | further anesthetic dispenser unit 38.1 into the |
| | supply fluid guide unit 30 |
| 100 | Ventilator, comprises the housing 12 with the |
| | receptacles 50, 50.1, the carrier gas mixer 24, |
| | the fluid conveying unit 4, the switching valve 20, |
| | the sensors 5.1, 5.2, 6.1, 6.2, 6.3, the fluid |
| | guide units 30, 3.1, 3.2 and the control unit 11 |
| Ag | Exhaust gas (excess gas mixture) is diverted |
| | from the ventilation circuit and extracted |
| | through connector 15 |
| Bg | Ventilation gas mixture, serves as the breathable |
| | gas mixture, comprises the anesthetic gas mixture |
| | Ng, Ng. 1 and the carrier gas Tg, is fed from |
| | the inspiratory fluid guide unit from the |
| | feed point 28 to the patient-side coupling unit 1 |
| Conₘₐₓ | Upper concentration threshold for the concentration |
| | of the anesthetic Nm in the anesthetic gas mixture |
| | Ng, at the same time upper limit of the value |
| | range from which the user can select a desired |
| | target concentration con_{req} with the |
| | aid of the control unit 26 |
| conₘₐₓ | Value of the upper concentration threshold Conₘₐₓ |
| | for the anesthetic gas mixture Ng |
| con.1ₘₐₓ | Value of the upper concentration threshold Conₘₐₓ |
| | for the anesthetic gas mixture Ng.1 |
| con_{req} | Target concentration of the anesthetic in the |
| | anesthetic gas mixture Ng, depends on a user |
| | specification, is at most equal to the value |
| | conₘₐₓ |
| con.1_{req} | Target concentration of the anesthetic in the |
| | anesthetic gas mixture Ng.1, depends on a user |
| | specification, is at most equal to the value |
| | con.1ₘₐₓ |
| min | Unit, calculates Tempₘᵢₙ = min (Temp_{amb}, Temp₃₈, |
| | Temp₅₀) |
| Ng | Anesthetic gas mixture, comprising the carrier |
| | gas Tg and the anesthetic Nm, is provided by |
| | the anesthetic dispenser unit 38 and fed to |
| | the feed point 28 by the supply fluid guide |
| | unit 30 |
| Ng.1 | Further anesthetic gas mixture, comprising the |
| | carrier gas Tg and the anesthetic Nm.1, is |
| | provided by the further anesthetic dispenser |
| | unit 38.1 and fed to the feed point 28 by the |
| | supply fluid guide unit 30 |
| Nm | Liquid anesthetic, comes from the anesthetic |
| | container 37 |
| Nm.1 | Liquid anesthetic, comes from the other |
| | anesthetic container 37.1 |
| ΔT | The time that has elapsed since the anesthetic |
| | dispenser unit 38 was inserted into the |
| | receptacle 50 is measured by the system clock 52 |
| Δt | Measured value of the time period ΔT |
| Temp_{amb} | Ambient temperature, measured by ambient |
| | temperature sensor 20 |
| temp_{amb} | Measured value of the ambient temperature |
| | Temp_{amb} |
| Temp₃₈ | Temperature of the anesthetic dispenser unit |
| | 38, is measured by the anesthetic dispenser |
| | temperature sensor 21 on the anesthetic |
| | dispenser side |
| temp₃₈ | Measured value of the temperature Temp₃₈ |
| Temp₅₀ | Temperature of the receptacle 50, is |
| | measured by the receptacle-side temperature |
| | sensor 22 on the receptacle side (ventilator |
| | side) |
| temp₅₀ | Measured value of the temperature Temp₅₀ |
| Tg | Carrier gas, is provided by the carrier |
| | gas mixer 24 and fed through the carrier gas |
| | fluid guide unit 49 to the anesthetic |
| | dispenser 36, 36.1 |
| Vol′_{3.1} | Volume flow through the first segment 3.1, |
| | measured by the first volume flow sensor 6.1 |
| Vol′_{3.2} | Volume flow through the second segment 3.2, |
| | measured by the second volume flow sensor 6.2 |
| Vol′₃₀ | Volume flow through the supply fluid guide |
| | unit 30, measured by the third volume flow sensor 6.3 |
| W | Wall, has the supply connection arrangement 13 |
| | and the socket outlet 16 |

What is claimed is:

1. A ventilation system for artificial ventilation of a patient, the ventilation system comprising:
a ventilator with a ventilator housing, which housing comprises a receptacle;
a patient-side coupling unit, which is configured to be arranged in and/or on the body of a patient to be artificially ventilated;
an anesthetic dispenser unit, which is at least temporarily inserted into the receptacle, the anesthetic dispenser unit being configured to generate an anesthetic gas mixture comprising an anesthetic;
a temperature sensor configured to yield a measured value of a measurable temperature, the temperature sensor being selected from a temperature sensor group consisting of an ambient temperature sensor, an anesthetic dispenser temperature sensor, and a ventilator temperature sensor, wherein the ambient temperature sensor is configured to measure a measurable ambient temperature in an environment of the ventilation system, wherein the anesthetic dispenser temperature sensor is configured to measure a measurable temperature at an anesthetic dispenser measuring position in or on or at the anesthetic dispenser unit, and wherein the ventilator temperature sensor is configured to measure a measurable temperature at a ventilator measuring position in or on or at the ventilator;
a data memory with a functional relationship that can be evaluated by a computer; and
a signal-processing control unit, which is configured to calculate a target concentration of the anesthetic in the anesthetic gas mixture to be generated, and to control the anesthetic dispenser unit with a control objective that the anesthetic dispenser unit generates the anesthetic gas mixture with the calculated target concentration,
wherein the ventilator is configured to convey a breathable gas mixture comprising oxygen and the generated anesthetic gas mixture to the patient-side coupling unit,
wherein the functional relationship specifies an upper concentration threshold, for the concentration of the anesthetic in the anesthetic gas mixture to be generated, as a function of the measurable temperature such that the upper concentration threshold increases with the measurable temperature increasing or at least remaining constant, wherein the measurable temperature is measured by the temperature sensor, and
wherein the control unit is further configured to apply the functional relationship to the measured value of the measurable temperature, to derive a value for the upper concentration threshold, and to calculate the target concentration such that the target concentration is at most equal to the derived value for the upper concentration threshold.

2. A ventilation system according to claim 1,
wherein the temperature sensor is the anesthetic dispenser temperature sensor,
wherein the ventilation system further comprises the ventilator temperature sensor,
wherein the functional relationship specifies the upper concentration threshold as a function of the temperature at the anesthetic metering position as the measurable temperature and of the temperature at the ventilator metering position as a further measurable temperature,
wherein the upper concentration threshold increases or remains constant with increasing temperature at the anesthetic dispenser measuring position while the temperature at the ventilator measuring position remains constant and wherein the upper concentration threshold increases or remains constant with increasing temperature at the ventilator measuring position while the temperature at the anesthetic dispenser measuring position remains constant, and
wherein the control unit is configured to apply the functional relationship to the measured value of a temperature at the anesthetic dispenser measuring position as the measured value and to a measured value of the temperature at the ventilator measuring position as a further measured value.

3. A ventilation system according to claim 2, wherein:
the anesthetic dispenser measuring position is in thermal contact with a surface of the anesthetic dispenser unit which surface faces the receptacle with the anesthetic dispenser unit being in an inserted state.

4. A ventilation system according to claim 2, wherein:
the ventilator measuring position is in thermal contact with a surface of the receptacle which surface faces the anesthetic dispenser unit being in an inserted state.

5. A ventilation system according to claim 1, further comprising an anesthetic sensor, which is configured to measure a concentration of the anesthetic in the generated anesthetic gas mixture, wherein the control unit is configured to control, using a signal from the anesthetic sensor, the anesthetic dispenser unit by a closed-loop control with a control objective that the actual anesthetic concentration in the generated anesthetic gas mixture is equal to the calculated target concentration.

6. A ventilation system according to claim 5, wherein the temperature sensor is the ventilator temperature sensor and the ventilator sensor temperature sensor at the ventilator measuring position is in thermal contact with the anesthetic sensor.

7. A ventilation system according to claim 1, further comprising:
an insertion sensor; and
a system clock,
wherein the ventilator is configured such that the anesthetic dispenser unit can be inserted into the receptacle and removed again from the receptacle,
wherein the temperature sensor is the ambient temperature sensor,
wherein the insertion sensor is configured to detect whether the anesthetic dispenser unit is inserted into the receptacle or not,
wherein the control unit is configured to measure, depending on a signal from the insertion sensor and depending on a signal from the system clock, an insertion time span which is a time span that has elapsed since a last insertion of the anesthetic dispenser unit into the receptacle,
wherein the measurable temperature of the functional relationship is the ambient temperature and the functional relationship specifies the upper concentration threshold additionally as a function of the insertion time span with the upper concentration threshold increasing or remaining constant with increasing insertion time span length if the ambient temperature remains constant, and
wherein the control unit is configured to apply the functional relationship to the measured value of the measurable ambient temperature and additionally to the measured value of the insertion time span.

8. A ventilation system according to claim 1,
wherein a set with two different possible anesthetics is given and wherein the functional relationship comprises for each possible anesthetic of the set a respective individual functional relationship,
wherein each individual functional relationship specifies the upper concentration threshold as a function of the measurable temperature,
wherein the upper concentration threshold increases with increasing measurable temperature or at least remains constant,
wherein the anesthetic dispenser unit is configured to generate an anesthetic gas mixture comprising one of the possible anesthetics, and
wherein the control unit is configured to calculate for each of the possible anesthetics a respective target concentration by applying the respective individual functional relationship for this anesthetic to the measured value of the measurable temperature, deriving a value for the upper concentration threshold, and calculating the target concentration such that the target concentration is at most equal to the derived value for the upper concentration threshold.

9. A ventilation system according to claim 8, wherein the control unit is configured to:
with the anesthetic dispenser unit inserted into the receptacle,
capture which anesthetic the anesthetic gas mixture generated by the anesthetic dispenser unit comprises;
select the individual functional relationship for the anesthetic captured; and
calculate the target concentration using the selected individual functional relationship.

10. A ventilation system according to claim 1,
wherein the control unit is configured to capture a specification,
wherein the captured specification comprises a target concentration and/or a target volume flow and/or a target mass flow of the anesthetic in the anesthetic gas mixture to be generated, and
wherein the control unit is further configured to derive and thereby calculate the target concentration of the anesthetic as a function of the captured specification.

11. A control arrangement for controlling a ventilation arrangement, wherein the ventilation arrangement is configured for artificial ventilation of a patient and comprises a ventilator with a ventilator housing, a patient-side coupling unit and an anesthetic dispenser unit, wherein the patient-side coupling unit is configured to be arranged in and/or on the body of a patient to be artificially ventilated, wherein the ventilator housing comprises a receptacle, wherein the anesthetic dispenser unit is at least temporarily inserted into the receptacle, wherein the anesthetic dispenser unit is configured to generate an anesthetic gas mixture comprising an anesthetic, and wherein the ventilator is configured to convey a breathable gas mixture comprising oxygen and the generated anesthetic gas mixture to the patient-side coupling unit, the control arrangement comprising:
a temperature sensor configured to yield a measured value of a measurable temperature, the temperature sensor being selected from a temperature sensor group consisting of: an ambient temperature sensor; an anesthetic dispenser temperature sensor; and a ventilator temperature sensor, wherein the ambient temperature sensor is configured to measure a measurable ambient temperature in an environment of the ventilation arrangement, wherein the anesthetic dispenser temperature sensor is configured to measure a measurable temperature at an anesthetic dispenser measuring position in or on or at the anesthetic dispenser unit, wherein the ventilator temperature sensor is configured to measure a measurable temperature at a ventilator measuring position in or on the ventilator;
a data memory with a functional relationship that can be evaluated by a computer and wherein the functional relationship specifies an upper concentration threshold for the concentration of the anesthetic in the anesthetic gas mixture as a function of the measurable temperature such that the upper concentration threshold increases with increasing measurable temperature or at least remains constant; and
a signal-processing control unit, which is configured to calculate a target concentration of the anesthetic in the anesthetic gas mixture to be generated, to control the anesthetic dispenser unit with a control objective that the anesthetic dispenser unit provides the anesthetic gas mixture with a calculated target concentration, wherein the control unit is further configured to:
apply the functional relationship to the measured value of the measurable temperature to derive a value for the upper concentration threshold; and
calculate the target concentration such that the target concentration is at most equal to the derived value for the upper concentration threshold.

12. A control process for controlling a ventilation arrangement, wherein the ventilation arrangement is configured to artificially ventilate a patient and comprises a ventilator with a ventilator housing, a patient-side coupling unit and an anesthetic dispenser unit, wherein the patient-side coupling unit is configured to be arranged in and/or on the body of a patient to be artificially ventilated, wherein the ventilator housing comprises a receptacle, wherein the anesthetic dispenser unit is at least temporarily inserted into the receptacle, wherein the anesthetic dispenser unit is configured to generate an anesthetic gas mixture comprising an anesthetic, and wherein the ventilator is configured to convey a breathable gas mixture comprising oxygen and the generated anesthetic gas mixture to the patient-side coupling unit, wherein the control process is carried with: a temperature sensor configured to yield a measured value of a measurable temperature, the temperature sensor being from a temperature sensor group consisting of: an ambient temperature sensor; an anesthetic dispenser temperature sensor; and a ventilator temperature sensor, wherein the ambient temperature sensor is configured to measure an ambient measurable temperature in an environment of the ventilation arrangement, wherein the anesthetic dispenser temperature sensor is configured to measure a measurable temperature at an anesthetic dispenser measuring position in or on the anesthetic dispenser unit, wherein the ventilator temperature sensor is configured to measure a measurable temperature at a ventilator measuring position in or on or at the ventilator, a data memory with a functional relationship that can be evaluated by a computer and wherein the functional relationship specifies an upper concentration threshold for the concentration of the anesthetic in the anesthetic gas mixture as a function of the measurable temperature such that the upper concentration threshold increases with increasing measurable temperature or at least remains constant, and a signal-processing control unit, the control process comprising the steps of:
with the temperature sensor, measuring the measurable temperature,
with the control unit, calculating a target concentration of the anesthetic in the anesthetic gas mixture to be generated, and
with the control unit, controlling the anesthetic dispenser unit with a control objective that the anesthetic dispenser unit provides the anesthetic gas mixture with a calculated target concentration,
wherein the step that the control unit calculates the target concentration comprises the steps that the control unit:
applies the functional relationship to the measured value of the measurable temperature and thereby derives a value for the upper concentration threshold; and
calculates the target concentration such that the target concentration is at most equal to the derived value for the upper concentration threshold.

13. A ventilation process for artificial ventilation of a patient, wherein the ventilation process is performed using a ventilation arrangement, wherein the ventilation arrangement comprises a ventilator with a ventilator housing, a patient-side coupling unit, an anesthetic dispenser unit configured to generate an anesthetic gas mixture comprising an anesthetic, a temperature sensor selected from a temperature sensor group, a data memory with a functional relationship that can be evaluated by a computer, and a signal-processing control unit, wherein the ventilator housing comprises a receptacle, wherein the functional relationship specifies an upper concentration threshold for a concentration of the anesthetic in the anesthetic gas mixture as a function of a measurable temperature such that the upper concentration threshold increases with increasing measurable temperature or at least remains constant, wherein the measurable temperature is an ambient temperature in an environment of the ventilation arrangement and/or a temperature at an anesthetic dispenser measuring position in or on or at the anesthetic dispenser unit and/or a temperature at a ventilator measuring position in or on the ventilator, wherein the ventilation process is performed while the patient-side coupling unit is arranged in and/or on the patient's body and the anesthetic dispenser unit is inserted into the receptacle, the process comprising the steps of:
with the temperature sensor, measuring the measurable temperature;
with the anesthetic dispenser unit, generating the anesthetic gas mixture comprising the at least one anesthetic;
with the ventilator, conveying a breathable gas mixture comprising the generated anesthetic gas mixture to the patient-side coupling unit;
with the control unit, calculating a target concentration of the anesthetic in the anesthetic gas mixture to be generated; and
controlling the anesthetic dispenser unit with a control objective that the anesthetic dispenser unit provides the anesthetic gas mixture with the calculated target concentration,
wherein the step that the control unit calculates the target concentration comprises the steps that the control unit:
applies the functional relationship to the measured value of the measurable temperature, thereby derives a value for the upper concentration threshold; and
calculates the target concentration such that the target concentration is at most equal to the derived value for the upper concentration threshold.
